(19) United States
(12) Patent Application Publication
(10) Pub. No.: US 2026/0314641 A1
(43) Pub. Date: Oct. 15, 2026

(54) PROVIDING NUTRITIONAL FORMULA TO A SUBJECT ADHERING TO A KETOGENIC DIET

(71) Applicant: N.V. Nutricia, Zoetermeer (NL)

(72) Inventors: Agustina Orlando, Buenos Aires (AR); Jacqueline Schuldberg, Buenos Aires (AR)

(73) Assignee: N.V. Nutricia, Zoetermeer (NL)

(21) Appl. No.: 19/646,644

(22) Filed: Apr. 14, 2026

Related U.S. Application Data
(63) Continuation of application No. PCT/EP2024/079385, filed on Oct. 17, 2024.

(30) Foreign Application Priority Data
Oct. 20, 2023 (EP) 23204930.4

(51) Int. Cl. A23L 7/10; A23L 29/269; A23L 33/00; A23L 33/115; A23L 33/125; A23L 33/19

(52) U.S. Cl. CPC A23L 7/198; A23L 29/27; A23L 33/115; A23L 33/125; A23L 33/19; A23L 33/40

(57) ABSTRACT

The invention pertains to a substantially gluten-free ketogenic flour blend having a ketogenic weight ratio between 2.3:1 and 2.9:1, preferably between 2.4:1 and 2.9:1, the blend comprising between 50 and 70 gram lipid per 100 g dry weight, comprising at least one, more preferably at least two, most preferably all of: (a) between 15 and 20 g MCT/100 g dry weight; (b) MCT providing 20-30% of the total energy content of the blend; (c) between 20 and 35 wt % MCT based on total weight of the lipid, and further comprising at least 2.5 g/100 g dry weight of fructo-oligosaccharides (FOS) and/or galacto-oligosaccharides (GOS), preferably FOS.

### FIELD OF THE INVENTION

The present invention is in the field of ketogenic treatment, particularly providing nutrition to a subject adhering to a ketogenic regimen.

### BACKGROUND OF THE INVENTION

The ketogenic diet (KD) is thought to result in adaptive changes to brain energy metabolism that increase the energy reserves. This is believed to help the neurons to remain stable in the face of increased energy demand and may confer a neuroprotective effect. In the ketogenic diet, carbohydrate levels are restricted. The principle of a KD, regardless of its form, is a limited carbohydrate intake with low glycemic response. Classical ketogenic diets [CKD] comprise an amount of lipids (weight), which is about 4-fold the weight of the sum of proteins and digestible carbohydrates. Fatty acids are used as the major source of fuel. These are used through fatty-acid oxidation in the cell mitochondria. The brain is normally fueled solely by glucose, lipids in the form of plasma lipoproteins as such do not cross the blood-brain barrier to a substantial degree. The liver can use long-chain fatty acids though to synthesize the three ketone bodies β-hydroxybutyrate, acetoacetate and acetone. These ketone bodies can under conditions enter the brain and act as a glucose substitute. Additionally medium-chain fatty acids octanoic (C8) and heptanoic (C7) acids can cross the blood brain barrier and be used by the brain. When glucose and ketone bodies are simultaneously present to neurons, glucose will by far be the preferred energy source. That is why the classical ketogenic diet has a large excess of lipids and hardly any digestible carbohydrates. Typically, the degree of ketogenicity decreases considerably when the amount of digestible carbohydrates in the diet is increased.

Since then, modifications to the classic KD have been developed intended to enhance compliance.

The Medium Chain Triglyceride Ketogenic Diet (MCTKD) is a variant thereof, unique because a percentage (30-70%) of total daily calories comes from medium chain triglycerides (MCTs). The idea of the MCTKD is that because MCTs produce more ketones than regular fat (which consists of mostly LCTs), incorporating high amounts of MCTs into the diet allows patients to stay in ketosis while consuming relatively higher amounts of protein and carbohydrates. Reference is made to Lin et al. “The ketogenic effect of medium-chain triacylglycerides” Frontiers in Nutrition (2021) vol. 9 article 747294. The original MCT diet developed in the 1970s derived 60% of its calories from MCT oil. Like CKD, MCTKD requires thorough calculation by dieticians. Prescription is not based on diet ratio but requires calculation of the percentage of total daily energy from LCT, MCT, protein, and carbohydrates. These can then be converted in grams of MCT oil and food choices and distributed into daily meal plans. As MCT oils produce higher levels of ketosis than LCTs, less total fat is needed in the MCT diet. Some 45-50% of energy from MCT is likely to achieve gastrointestinal tolerance and level of ketosis comparable with CKD, but can range from 30-70% based on an individual patient's tolerance. MCT oil can also be used as a supplement to CKD meals to boost ketosis or to treat hyperlipidemia and constipation attributable to CKD. However, consuming large quantities of MCT oil causes abdominal cramps, diarrhea and vomiting though, which is a severe drawback for its use. The diet is, like the ketogenic diet, unpalatable which makes it difficult to adhere with. In addition, the health consequences of a very high fat diet for life can be considerable.

The Low Glycemic Index Diet is another variant of the classical KD. The glycemic index classifies carbohydrates by their potential to raise blood glucose on a scale from 0-100. The low glycemic index diet allows for a greater intake of digestible carbohydrates, but the carbohydrates are limited to those with a low glycemic index (typically considered a glycemic index of 55 or less). In practice low GI nourishment does not induce a ketogenic state and people find the diet often difficult to adhere to. The Modified Atkins Diet (MAD) is another, more liberal variation of the traditional ketogenic diet mainly limited in the amount of digestible carbohydrates. While the ketogenic diet is typically given at 4:1 or 3:1 ketogenic ratio, which is the ratio of the amount of fat to the combined amounts of protein and digestible carbohydrates, MAD involves about a 1:1 ratio, which allows for more protein and carbohydrate intake, and requires less fat intake. However, the drawback of the MAD is that can be difficult for patients on the MAD to comply with and to get enough fat to remain in ketosis.

A modified ketogenic diet (MKD) is a hybrid KD, adopting principles from other established KD protocols and defining new elements to the MKD. It was designed to find optimal balance between ketosis and palatability in particular patients. In fact, presently the majority of KDs are individualized MKDs, differing from each other, tailored to individual patients' needs (energy requirements, macronutrients allowance) in order to find optimal equilibrium between efficacy and adherence. It is perceived as less restrictive and more palatable in comparison to CKD and may be associated with a greater compliance by older children, teenagers, and adults. MCTKD has similar efficacy to the CKD, but in comparison permits proportionally less total fat and more carbohydrate and/or protein to be included making it more palatable especially for children with limited food options.

However, as addressed in Zarnowska “Therapeutic use of the ketogenic diet in refractory epilepsy: what we know and what still needs to be learned” Nutrients (2020), 12, 2616, despite numerous attempts to reduce the restrictiveness and prescriptiveness of the KD, it remains a challenging task for both the patients and/or caregivers and the keto-team. Even the alternative ways generating a nutritional ketosis state which have been developed in the last 20 years and which changed the prescription rules (calories, ratio of fat to carbohydrate, quality and quantity of macronutrients content, methods of diet implementation and maintenance) struggle with adherence to ketogenic therapy. Despite the evidence of medical ketogenic diet regarding its effectiveness on seizures control for refractory patients, adherence and compliance to the strict types of diet is one of the higher burdens.

The diets have a perceived relative complexity which by itself already leads to adherence problems. When patients are tube fed, compliance and effectiveness are higher than for patients that consume orally. Because high fat ratios and carbs restrictions, usual meals become difficult. For families, carers and patients, palatability and easy-to-prepare options are key factors to be encouraged to pursuit long term treatment. Both children and adults struggle with adherence, and palatability is key to diminish dropouts. The effectiveness of the treatment is jeopardized by the feasibility of long-term adherence. It is particularly the strong prevalence of fats, in particular triglycerides, that can be detrimental to the digestibility of the products. There are flours commercialized for ketogenic patients to make baked or boiled food products based on 4:1 ketogenic weight ratios, but which are associated with the above disadvantageous sensory attributes. On the other hand, US2023/021730 describes a very low ratio, between 1.5:1 and 2.5:1, and advocates the use of large amounts of MCTs, from 35-95% to 80-100%. However, the present inventors found that such low ketogenic formulations are not well appreciated because the fat and nutrients balance is less optimal and the products baked and boiled with such a fat profile often show consistency issues. The high MCT concentrations may well lead to gastrointestinal intolerance, and give the product an oily characteristic which hinders the application in making a good dough (which remains together) and baking foods with it. With these kinds of compositions, it remains a challenge to make pasta and boiled foods.

Hence, there continues to be a need for ketogenic products which allow easier preparation and maintenance, and improved palatability and tolerability, thus improving adherence or compliance with the ketogenic treatment.

### SUMMARY OF THE INVENTION

The inventors have developed an easy-to-handle MCT-comprising substantially gluten-free ketogenic flour blend based on a mixture of high-fat flours, MCT powder and fructo-oligosaccharides, to yielding a versatile range of baked or boiled food products with improved palatability and crunchiness, suited for use in a ketogenic regimen, and boosting treatment compliance. The ingredients of the formula have been carefully selected to ultimately yield baked and boiled products with improved flavour; texture; color, appearance/appeal; easy to prepare/work with; crunchiness; dry/not oily aftertaste, yet while retaining a ketogenic weight ratio so desired. In ketogenic flours (aka keto flours); all-purpose (gluten-containing) wheat flours which are common baking ingredients to healthy people are carefully avoided, since these typically have about 60% of their dry weight formed from carbohydrates. The ingredient blend of the invention comprises reduced amounts of (gluten-free) buckwheat flour and provides key nutrients for subjects on a ketogenic diet with organoleptic characteristics which are improved from prior art ketogenic diet, with the aim to improve quality of life and adherence to the diet. The flour blend of the invention can still serve as an all-purpose flour in a ketogenic regimen, and can particularly be used to make homemade-like baked food products with a crunchy appearance, texture and taste otherwise associated with gluten-containing wheat flour-based baking, wherein the specific blend of ingredients and mixture of substantially gluten-free high-fat flours makes up for the loss of the binding and structuring powers of gluten which is found in common wheat flour. In sensory panel testing, baked products based on the flour according to the invention stood out and were much better appreciated in terms of sensory attributes, in particularly in terms of texture, than those based on commercial flours with 4:1 ketogenic ratio. Also, the inventors provided a shelf-stable composition which keeps its appearance (odor, color etc.) for at least 8 months. Reference is made to the examples.

Thereto, the invention relates to a substantially gluten-free ketogenic flour blend having a ketogenic weight ratio (i.e. the sum of the weight of all lipid over the sum of the weight of all protein and digestible carbohydrates) between 2.3:1 and 2.9:1, further comprising at least 2.5 g/100 gram fructo-oligosaccharides. Constipation is one of the most common side effects of medical ketogenic diet. Despite this observation, there are no official dietary recommendations for the “adequate intake” or “recommended daily allowance” of prebiotics in healthy individuals, most gut prebiotics require an oral dose of at least 3 grams per day or more to confer benefit.

More particularly, the flour blend comprises between 50 and 70 wt % gram lipid, based on dry weight of the blend. It is preferred that the blend is further characterized by at least one, more preferably at least two, most preferably all of: (a) between 15 and 20 g MCT/100 g dry weight; (b) MCT providing 20-30%, preferably 20-25% of the total energy content; (c) between 25 and 35 wt % MCT based on total weight of the lipid fraction. More particularly, the flour blend comprises between 50 and 70 wt % gram lipid, based on dry weight of the blend, wherein 25-35 wt % of the lipid fraction is provided by MCT and/or wherein 20-30 en % of the total caloric content of the blend is provided by MCT. The flour is an all-purpose flour, but particularly suitable for preparing baked or boiled food products such as bread, cake, muffin, biscuit, cookie, scone, pasta, and sweet or salty pies, and the like. With the flour blend of the invention the subject can be provided with home-made, ketogenic baked food products with the texture (crispy, crunchy, or fluffy) that is associated with the same non-ketogenic baked food product counterpart.

The blend preferably comprises a mixture of almond flour and/or coconut flour, and buckwheat flour. In one embodiment, there is less than 50% buckwheat flour based of all flour ingredients comprised in the blend. The mixture reduces the amount of carbohydrates in the blend compared to the conventional gluten-containing wheat flour counterpart (which has substantive amounts of carbohydrates).

The invention particularly relates to a ketogenic flour blend suitable for the purpose of preparing baked or boiled food products, preferably substantially gluten-free, having a ketogenic weight ratio between 2.3:1 and 2.9:1, comprising between 50 and 70 gram lipid per 100 g dry weight, wherein 20-35 wt % of the lipid fraction is provided by MCT and/or wherein 20-30 en % of the total caloric content of the blend is provided by MCT, and further comprising at least 2.5 g/100 g dry weight of galacto-oligosaccharides and/or fructo-oligosaccharides, preferably fructo-oligosaccharides. The ketogenic weight ratio range allows for a 10% variation on the ratio of 2.6:1 that was proven successful in the accompanying examples.

### LIST OF PREFERRED EMBODIMENTS

1. A substantially gluten-free ketogenic flour blend having a ketogenic weight ratio between 2.3:1 and 2.9:1, comprising between 50 and 70 gram lipid per 100 g dry weight, comprising at least one, more preferably at least two, most preferably all of: (a) between 15 and 20 g MCT/100 g dry weight; (b) MCT providing 20-30% of the total energy content of the blend; (c) between 20 and 35 wt % MCT based on total weight of the lipid, and further comprising at least 2.5 g/100 g dry weight of fructo-oligosaccharides (FOS) and/or galacto-oligosaccharides (GOS), preferably FOS.

2. The flour blend according to embodiment 1, having a ketogenic weight ratio between 2.4:1 and 2.8:1.

3. The flour blend according to embodiment 1 or 2, comprising a mixture of almond flour and/or coconut flour, and buckwheat flour, preferably comprising less than 0.5 wt % flaxseed flour, based on the total dry weight of the blend.

4. The flour blend according to any one of the preceding embodiments, comprising protein and digestible carbohydrates in a weight ratio between 1.2:1 and 1:1.2.

5. The flour blend according to any one of the preceding embodiments, comprising between 55 and 65 wt % lipid, based on total dry weight.

6. The flour blend according to any one of the preceding embodiments, comprising 20-35 wt %, more preferably 20-30 wt %, even more preferably 25-30 wt % MCT, based on total weight of the lipid fraction.

7. The flour blend according to embodiment 6, wherein the MCT provide 20-30 en % of the total caloric content of the blend.

8. The flour blend according to any one of the preceding embodiments, comprising 3-5 g/100 g dry weight of GOS and/or FOS, preferably FOS.

9. The flour blend according to any one of the preceding embodiments, providing 6.0-7.0 kcal/100 g dry weight of the blend.

10. The flour blend according to any one of the preceding embodiments, comprises 7-15 g dietary fibers, preferably 8-14 g, more preferably 10-12 g fibers per 100 g of the ketogenic blend.

11. The flour blend according to any one of the preceding embodiments, comprising 10-20 g of protein per 100 g, preferably 10-15 g of protein per 100 g of the blend; and/or wherein the protein provides 5-10 en % of the total energy content of the ketogenic blend.

12. The flour blend according to any one of the preceding embodiments, comprising 10-20 g of digestible carbohydrates per 100 g, preferably 10-15 g of digestible carbohydrates per 100 g of the blend; and/or wherein the digestible carbohydrates provide 5-10 en % of the total energy content of the ketogenic blend.

13. The flour blend according to any one of the preceding embodiments, comprising casein and whey protein, almond flour, coconut flour, buckwheat flour, coconut milk, fructo-oligosaccharides, Psyllium, xanthan gum, resistant starch and albumin.

14. The flour blend according to any one of the preceding embodiments, providing, per 100 g,

| | 667 kcal/2604 kJ | |
|---|---|---|
| digestible carbohydrates | 11 | g |
| total sugar | 2.3 | g |
| added sugar | 2.1 | g |
| Protein | 12 | g |
| total fat: | 60 | g |
| polyunsaturated | 10 | g |
| linoleic acid (n6) | 8.5 | |
| α-linolenic acid (n3) | 0.9 | |
| Saturated | 35 | g |
| MCT, of which: | 17 | |
| caprylic acid (C8) | 9.8 | |
| capric acid (C10) | 6.2 | |
| Monounsaturated | 15 | g |
| Fibre: | 11 | g |
| soluble, | 4 | g |
| of which FOS | 3.4 | |
| Insoluble | 7 | |

allowing for a 10% variation in the above numbers, more preferably a 5% variation in the above numbers in the table, provided that the sum of carbohydrates, protein, fat and fibre amounts to 100%, and the blend has a ketogenic weight ratio between 2.4:1 and 2.8:1, and wherein the blend comprises a mixture of almond flour and/or coconut flour, and buckwheat flour, the latter preferably in an amount of less than 50% of all flour ingredients comprised in the blend.

15. A method for producing a bakery product, comprising providing a dough by mixing the flour blend according to any one of the preceding embodiments with egg, butter and baking powder according to the desired bakery product recipe, and subjecting the dough to baking.

16. A bakery product suitable for a ketogenic diet and obtainable by the method according to embodiment 15, preferably having a ketogenic weight ratio between 2.4:1 and 3.0:1, more preferably between 2.6:1 and 3.0:1.

17. A method for providing nutrition to a human subject adhering to a ketogenic diet regimen, comprising administering to the patient a bakery product according to embodiment 16.

18. A substantially gluten-free ketogenic flour blend according to any one of embodiments 1-14, for use in the treatment of epilepsy and/or prevention of progression of epilepsy, for use in the treatment and/or prevention and/or reduction of the severity of epilepsy and in prevention and/or delay of the formation of epileptiform circuits in the brain, and for use in preventing abnormal elevations of blood triglyceride levels, fatty liver development and/or metabolic syndrome development in the human patient adhering to a ketogenic diet.

19. Use of the ketogenic blend according to any one of embodiments 1-14 in the manufacture of a baked or boiled food product for a human subject adhering to a ketogenic diet, wherein the baked or boiled food product has a ketogenic weight ratio between 2.3:1 and 3.0:1, preferably between 2.6:1 and 3.0:1, preferably a human patient which is at increased risk of or suffering from (drug-resistant) epilepsy.

20. A substantially gluten-free ketogenic flour blend according to any one of embodiments 1-14, for use in enhancement of mitochondrial function and reduced use of glucose as a metabolic substrate, particularly Alzheimer's disease, Parkinson's disease and Amyotrophic lateral sclerosis, ADHD.

### DETAILED DESCRIPTION OF THE INVENTION

The terms ‘ketogenic blend’ and ‘ketogenic composition’ in the context of the invention are used interchangeably, and mean to cover nutrition, a nutritional formula or nutritional composition that is suitable for ketogenic subjects, and particularly suitable for manufacturing nutrition for oral administration to ketogenic subjects, or human subjects adhering to a ketogenic diet.

The invention relates to a ketogenic flour blend meaning that the blend comprises an amount of lipids (by weight), which is between 2.3 and 2.9 times the weight of the sum of proteins and digestible carbohydrates comprised in the blend. It is preferred that the ketogenic flour blend has a so-called ketogenic weight ratio between 2.4:1 and 2.9:1, more preferably between 2.4:1 and 2.8:1 (allowing for a 5% variation around the successful 2.6:1 ketogenic ratio in the accompanying examples). The composition comprises at least one, more preferably at least two, most preferably all of: (a) between 15 and 20 g MCT/100 g dry weight; (b) MCT providing 20-30%, preferably 20-25% of the total energy content; (c) between 20 and 35 wt % MCT based on total weight of the lipid fraction. In the context of the invention, the ketogenic (weight) ratio is the weight ratio of the weight amount of lipid to the combined (sum) weight amounts of protein and digestible carbohydrates in the composition. In the context of the invention, where reference is made to the “total weight of the ketogenic composition” this refers to the sum of the weights of the ingredients forming the ketogenic composition without the addition of water, i.e., the water-free or dry weight. Within the aforementioned (sub) ranges of ketogenic ratios of the invention, the ketogenic diet used within the context of the invention preferably comprises a lipid content that is at least twice the carbohydrate content by weight. The ketogenic flour blend is substantially gluten-free, more preferably gluten-free.

In the context of the invention a subject adhering to or taking a ketogenic diet is a human. It is particularly directed to human subjects, preferably human patients at increased risk of or suffering from (drug-resistant) epilepsy, convulsions or epileptic seizures, preferably to prevent progression of epilepsy in a subject suffering from epilepsy or at risk of seizures.

The blend is substantially gluten-free meaning that the ketogenic blend is substantially free from any gluten-containing ingredients such as wheat flour; it is preferred that the ketogenic blend comprises, if at all, less than 200 ppm gluten, more preferably less than 100 ppm, based on the total weight of the blend. In a most preferred embodiment, the blend is gluten-free; taking the FDA guidelines as a base, ‘gluten-free’ in the context of the invention means that the blend contains less than 20 parts per million (ppm) of gluten; this level is the lowest that can be reliably detected in foods using scientifically validated analytical methods. The gist is that the blend is free from wheat flour which is a source of gluten and carbohydrates. A possible example of a gluten-free baked food product is gluten-free bread. It is believed that there is an association between gluten sensitivity and epilepsy or seizures, therefore the gluten-free composition of the invention would bring further benefits.

The flour blend preferably comprises a mixture of almond flour and/or coconut flour, and buckwheat flour. In itself, buckwheat flour is not known to be ‘keto-friendly,’ but the combination with almond and coconut flour provides for a flour blend with improved ease of handling and palatability, provided the amount of buckwheat flour is selected for the blend to remain within the desired ketogenic weight ratio according to the invention. In one embodiment, the flour blend comprises a mixture of coconut flour and buckwheat flour, advantageously free from almond allergen. The mix of flours in the blend of the invention is significantly reduced in carbohydrates compared to conventional wheat flour. Although flaxseed flour is also known to be low in carbohydrates, the blend is preferably low or free from flaxseed flour; if any, there is preferably less than 0.5 wt % flaxseed flour, more preferably less than 0.1 wt % flaxseed flour, based on total weight of the blend. It was found that elimination of flaxseed improves the appeal of the product. In the experiments, the use of flaxseed oil resulted in black spots in the final mixture.

The total amount of lipids that is present in the ketogenic composition, i.e., the combination of all lipids (including fat and oil) and phospholipids present, is referred to as the ‘lipid fraction’ of the ketogenic composition. Lipids are understood here in the sense commonly used in the state of the art, with particular reference to the definition by IUPAC (International Union of Pure and Applied Chemistry) of “substances of biological origin soluble in non-polar solvents” (source: PAC, 1995, 67, 1307, “Glossary of class names of organic compounds and reactivity intermediates based on structure”, IUPAC Recommendations 1995, page 1348). Suitable sources of lipids to provide for the lipid fraction of the ketogenic composition according to the invention are food-grade ingredients, for example those derived from eggs, milks, vegetables or plant material, marine organisms like fish or algae, natural- or mutated organisms or the lipids prepared by modification of the natural lipids, processed lipids from these sources such as extracts, isolates, inter-esterified lipids and the like, synthetic compounds or combinations thereof. Throughout this application the amounts of lipids are, unless otherwise indicated such as for example when reference is made to MCTs, described in terms of the molar weight of the fatty acids (Fas) present in the composition and corrected for the weight of glycerol in an oil blend. The lipid fraction of the ketogenic composition contains a mixture of medium chain, long chain, and very long chain saturated fatty acids, as well as long chain and very long chain mono- and poly-unsaturated fatty acids. The fatty acids may be present in the ketogenic composition in either free form or bound either combined or non-combined to glycerol as mono-, di- or triacylglycerols and bound to any of the sn-1, sn-2 or sn-3 position of glycerol. Fatty acids may be either unsaturated or saturated. Fatty acids which are not attached to other molecules are referred to as free fatty acids (FFA).

In one embodiment the lipid fraction preferably provides up to 90% of the total amount of calories (en %) of the ketogenic composition, more preferably 70-90% of the total amount of calories, more preferably 75-85% of the calories. The remainder of the calories of the ketogenic composition is provided by carbohydrates, proteins, and fibers. The energy provided by the nutrients in the blend is calculated using the Atwater calculation factors of 9 kcal per g lipid, 4 kcal per gram protein or gram digestible carbohydrate, 2 kcal per gram dietary fiber and zero kcal for the other components in the product. Alternatively, or additionally, the amount of lipid in the ketogenic blend may be characterized in terms of the weight contribution to the product. The ketogenic composition preferably comprises 50-70 g lipid per 100 g, more preferably 55-65 g lipid per 100 g.

The amount of lipid can be determined by applying methods known in the art for measuring fat content in a food matrix as applicable. For example, fat content for general foods is determined by applying AOACI official method 983.23, while the Roese-Gottlieb method (AOACI 932.06) is better applicable for products based on dried milk (Lehner, R., Estoppey, A., (1954) Mitt. Lebensmitteluntersuchung Hyg. 54:183-185). The amount of individual lipid components can be determined by applying methods specifically designed for measuring that specific component or by fractionating the fat fraction isolated from the extraction of the chloroform-methanol fraction as given in the 983.23 method. Additionally, the structural composition of the lipids such as the binding at the sn-1, sn-2 or sn-3 position to form mono-, di- or tri-acylglycerols may be determined and quantified using liquid chromatography, mass spectrometry (also called lipidomics) and other methods known in the art (Beermann C et al., Lipids, 2005; 40(2): 211-8).

The ketogenic composition according to the invention comprises saturated medium chain fatty acids [MCFAs]. Preferably at least 40 wt % of the MCFAs are provided in the form of MCTs. MCTs are triglycerides derived from saturated MCFAs which have aliphatic chains of carbon with chain lengths of 6 (hexanoic acid), 8 (caprylic or octanoic acid), 10 (capric or decanoic acid) and 12 (lauric or dodecanoic acid) carbon atoms. In the blend, it is preferred that 25-35 wt % of the lipid fraction is provided by MCT and/or wherein 20-30 en % of the total caloric content of the blend is provided by MCT.

For example, medium-chain fatty acids with 10 carbon atoms may be referred to herein as “C10 fatty acids” or “C10”. The MCT fraction in the ketogenic composition is beneficially rich in C8:0, C10:0 and C12:0 fatty acids (FA), wherein “rich” preferably means that these FAs together forming more than 70 wt % of the FAs in the MCT fraction, preferably more than 80 wt % of the MCT fraction. In some aspects up to 100 wt % of the FAs in the MCT fraction are C8:0, C10:0 and C12:0 fatty acids. It is believed that the provision of an MCT blend rich in C8:0, C10:0 and C12:0 fatty acids aids in the ketogenicity of the diet. When calculated in terms of MCTs, the MCT preferably provides 20-30%, preferably 20-25% of the total energy content of the ketogenic composition. In a further embodiment the lipid fraction of the ketogenic composition preferably comprises 25-35 wt % MCTs based on total weight of the lipid fraction.

In one embodiment, the MCTs that can be used in the present invention are particularly rich in triglycerides derived from octanoic acid (C8:0), decanoic acid (C10:0), or combinations thereof, although other substances can be present, in the form of traces or impurities, such as for example caproic acid (C6:0), heptanoic acid (C7:0), lauric acid (C12:0), wherein “rich” is preferably defined as C8:0 and C10:0 together forming more than 80 wt %, more preferably more than 90 wt % of the saturated MCFAs in the MCT fraction of the MCT fraction. In some aspects up to 100 wt % of the FAs in the MCT fraction are C8:0 and C10:0 fatty acids.

In one embodiment, the sum of the weight of medium chain fatty acids C8:0+C10:0 over C12:0 in the ketogenic composition is more than 3:1, more preferably more than 4:1, even more preferably more than 5:1.

In a preferred embodiment, the MCT comprises reduced amounts of C6:0. Although it is a known ketogenic and neuroprotective fatty acid, it contributes to the taste in a negative way, and is associated with gastrointestinal issues. Hence, it is preferred that the amount of C6:0 is less than 5 wt %, more preferably less than 1 wt % of the saturated fatty acids is C6:0 fatty acids.

MCTs are a type of fat found in a small number of foods, such as coconut oil and palm kernel oil. MCTs may be supplied by commercially available MCT oils. MCFAs are defined to be linear or branched, preferably linear, saturated carboxylic acids having six to twelve carbon atoms. When bound to a glycerol backbone at the sn-1 to sn-3 positions of glycerol MCFAs are present in the form of MCTs. Within the scope of the invention, MCTs also include glycerides wherein at least one of the FAs is an MCFA, preferably at least 2, most preferably 3 of the FAs are MCFAs. The presence of MCTs in the ketogenic composition is very advantageous, since they are effective in promoting a rapid induction of ketosis, that is, a rapid production and secretion of ketones by the liver into the blood circulation to create a condition of ketosis. In addition, MCTs are particularly beneficial for neurons for suppressing seizures and also induce the local production of ketones by glial cells (astrocytes) and supply to neurons in the brain.

As used in the context of the invention, the term “ketosis” as used herein refers to a subject having blood ketone levels above 0.5 mmol/L. Ketone levels sustained above 0.5 mmol/L and ideally in the range of 1.0 to 3.0 mmol/L appear to offer therapeutic effects in humans. Levels of ketones in the blood above 10.0 mmol/L are associated with signs of ketoacidosis. While ketosis refers to a state of elevated ketones, ketoacidosis is a pathological and potentially life-threatening condition amongst others resulting in a decrease in blood pH and may induce a coma. Means to detect blood and/or urine ketone levels and or blood pH are known in the art.

The ketogenic composition preferably comprises omega-3 and/or omega-6 polyunsaturated fatty acids (PUFA).

The ketogenic composition comprises digestible carbohydrates, preferably digestible carbohydrates with a low glycemic index. The blend preferably comprises 10-20 g of digestible carbohydrates per 100 g, preferably 10-15 g of digestible carbohydrates per 100 g of the blend; and/or the digestible carbohydrates provide 5-10 en % of the total energy content of the ketogenic blend. In a preferred embodiment the glycaemic index (GI) of the ketogenic composition is below 70, preferably below 65, preferably below 60, more preferably below 55. In a preferred aspect the glycaemic index is between 65 and 25, wherein the glycaemic index is based on glucose as reference value set at 100. Typically, any digestible carbohydrates that are known in the art to be suitable for use in nutritional compositions may be used. Preferably, the ketogenic composition comprises digestible carbohydrates rich in one or more of non-glucose and non-fructose monosaccharides such as mannose, galactose, xylulose, xylose, glucosamine, and sialic acid. In a preferred embodiment the composition according to the invention comprises one or more low glycemic index carbohydrates selected from trehalose, lactose, galactose and isomaltulose, more preferably galactose and isomaltulose. Typically, such low GI carbohydrates are carbohydrates having a GI of 55 or less. Low glycemic index carbohydrates beneficially allow the inclusion of carbohydrates in the ketogenic composition while not significantly impacting ketogenesis.

The ketogenic composition also comprises non-digestible carbohydrates or dietary fibers, comprising at least FOS or GOS, preferably FOS. Since the ketogenic diet, particularly a gluten-free ketogenic diet, is known to disturb the microfloral balance in the intestines, the addition of these dietary fibers, particularly prebiotic oligosaccharides such as GOS and FOS, brings further benefit by reducing gastrointestinal issues associated with the ketogenic diet. Fibres are non-digestible carbohydrates. Non-digestible carbohydrates are carbohydrates that are resistant to digestion and absorption in the human stomach and small intestine and enter the colon intact. So, compounds like lactose, maltose, glucose, (regular, unmodified) maltodextrin, and (regular, unmodified) starch are regarded as digestible. Fibres can be fermentable in the colon, or non-fermentable. The term “fermentable” refers to the capability to undergo (anaerobic) breakdown by micro-organism in the lower part of the gastro-intestinal tract, e.g. colon, to smaller molecules, in particular short chain fatty acids and lactate. The fermentability may be determined by the method described in Am. J. Clin. Nutr. 53, 1418-1424 (1991). The ketogenic composition preferably comprises 7-15 g dietary fibers, preferably 8-14 g, more preferably 10-12 g fibers per 100 g of the ketogenic composition. Worded differently, the ketogenic composition preferably comprises 7-15 wt % fibers, preferably 8-14 wt %, more preferably 10-12 wt % of dietary fibers based on total weight of the ketogenic composition.

The non-digestible carbohydrates or dietary fibers are preferably galacto-oligosaccharides and/or fructo-oligosaccharides (including fructo-polysaccharides such as inulin). Preferably at least 50 wt % of these non-digestible oligosaccharides have an average degree of polymerization of 2 to 60. In one embodiment, the composition comprises at least galacto-oligosaccharides, in particular at least beta-galacto-oligosaccharides, more in particular at last trans-galacto-oligosaccharides. The galacto-oligosaccharides preferably comprise saccharides with an average DP of 2 to 10. (Trans) galactooligosaccharides are for example available under the trade name Vivinal® GOS (Borculo Domo Ingredients, Zwolle, Netherlands), Bimuno (Clasado), Cup-oligo (Nissin Sugar) and Oligomate55 (Yakult).

The non-digestible carbohydrates preferably comprise fructooligosaccharides. Fructooligosaccharides may be inulin hydrolysate products having an average degree of polymerization (DP) between 2 and 200. FOS products are for instance commercially available as Raftilose P95 (Orafti) or Beneo® P95. Long-chain fructooligosaccharide is preferred, preferably having an average DP between 10 and 60, more preferably between 15 and 50. A particular type of long-chain fructo-oligosaccharides is inulin, such as Raftilin HP. The amount of FOS in the blend is preferably between 2.5 and 5 wt % of the dry weight, even more preferably between 3 and 4 wt %. It is preferred that FOS provides for 25-40% of the total amount of soluble and non-soluble fiber in the blend.

In addition to the non-digestible oligosaccharides, the fibers of the composition preferably comprise xanthan gum and psyllium. Both are ‘add-ons’, not essential to realize the organoleptic and textural improvements according to the invention, but traditionally used in products for ketogenic diets and conveniently added here.

In a preferred embodiment the composition further comprises insoluble fibres like resistant starch and/or cellulose, preferably resistant starch, preferably an RS4-type starch. It is preferred that the non-soluble fibres represent at least 50 wt % of the total amount of fibers in the ketogenic composition. The ketogenic composition according to the invention provides about 2 kcal per g fiber and preferably provides 2-5% of the total amount of calories of the ketogenic composition.

The ketogenic composition according to the invention contains a protein source. Protein includes all proteinaceous material, including intact and (partly) hydrolysed protein, peptides and free (added) amino acids. The total protein that is present in the ketogenic composition, i.e., the combination of all proteins and amino acids present, may be referred to as the “protein fraction” of the ketogenic composition. Any source of protein suitable to be used in a nutritional composition may be comprised in the ketogenic composition according to the invention. Preferred proteins sources include dairy proteins such as whey, casein, vegetables (beans, pea, soy, lupin, amaranth, potato, seed, grains tubers, etc.), fish, krill, algae, animals' proteinaceous material, eggs, and mushrooms. In an embodiment the ketogenic composition preferably comprises 10-20 g of protein per 100 g of the composition, preferably 10-15 g of protein per 100 g of composition. Additionally, or alternatively, in an embodiment the protein provides 5-10 en % of the total energy content of the ketogenic composition. Advantageously, this protein content allows to reach high ketogenic ratios, making the ketogenesis of the present food formulation and of the present food product more effective. In one embodiment the protein fraction comprises at least 75 wt % intact and/or (partly) hydrolysed protein, based on total weight of the protein fraction. In a preferred embodiment the protein fraction comprises intact and/or (partly) hydrolysed protein selected from casein and/or whey, preferably casein. In one embodiment, the protein is supplied as albumin.

In a preferred embodiment the ketogenic composition further comprises citrate. Citrate as used in the neuroprotective composition may be in the form of citric acid or a salt of potassium (K+), sodium (Na+), Magnesium (Mg2+) or calcium (Ca2+). Citrate or citric acid is converted to isocitrate in the first step in the Krebs cycle (also known as the citric acid or TCA cycle). Citrate was found to increase glycolytic rates in in vitro neuroglial models and provide prevention against neuronal damage by increasing energy production, preferably through a coupled increase in glycolysis in glial cells and mitochondrial oxygen consumption in neurons in the brain. Citrate is believed to reduce and/or prevent excitotoxity associated with or following seizures, and consequently prevent and/or treat neuroinflammation associated with epileptic seizures, and consequently prevent and/or delay the formation of epileptiform circuits in the brain. In a preferred embodiment the ketogenic composition comprises 100-1000 mg citrate per 100 g, more preferably 250-750 mg citrate, even more preferably 500-600 mg citrate per 100 g of the ketogenic composition. Worded differently, the ketogenic composition preferably comprises 0.1-1 wt % citrate, more preferably 0.25-0.75 wt %, even more preferably 0.5-0.6 wt % based on total weight of the ketogenic composition. Any amounts of citrate equivalents or derivatives are calculated to the equivalent weight amount of citrate, counterions excluded.

In an embodiment, the gluten-free ketogenic composition provides, per 100 g,

| | 667 kcal/2604 kJ | |
|---|---|---|
| digestible carbohydrates | 11 | g |
| total sugar | 2.3 | g |
| added sugar | 2.1 | g |
| Protein | 12 | g |
| total fat: | 60 | g |
| polyunsaturated | 10 | g |
| linoleic acid (n6) | 8.5 | |
| α-linolenic acid (n3) | 0.9 | |
| Saturated | 35 | g |
| MCT, of which: | 17 | |
| caprylic acid (C8) | 9.8 | |
| capric acid (C10) | 6.2 | |
| Monounsaturated | 15 | g |
| Fiber: | 11 | g |
| soluble, | 4 | g |
| of which FOS | 3.4 | |
| Insoluble | 7 | , |

allowing for a 10% variation in the above numbers, more preferably a 5% variation in the above numbers in the table, provided that the sum of carbohydrates, protein, fat and fiber amounts to 100%, and the blend has a ketogenic weight ratio between 2.4:1 and 2.9:1, more preferably between 2.4:1 and 2.8:1, even more preferably between 2.5:1 and 2:7:1, and wherein the blend comprises a mixture of almond flour and/or coconut flour, and buckwheat flour, the latter preferably in an amount of less than 50% of all flour ingredients comprised in the blend. It is preferred that the blend comprises 6-9 wt % of the sum of almond flour and coconut flour, and 1-3 wt % buckwheat flour; more preferably 3-6 wt % of each of almond flour and coconut flour, and 1-3 wt % buckwheat flour.

The flour blend is suitable for baking purposes i.e., making baked or boiled food products. A baked or boiled food product can be understood as a baked or boiled food product based on flour, cereals or not cereals, such as bread, bread substitutes, such as crackers, croutons, breadsticks, or desserts, sweet or savory pies, donuts, panettone, pandoro, unleavened bread, piadinas, tortillas, pizzas, focaccias, biscuits, pastry products, croissants, brioches, puff pastry in general, or suchlike. This includes baked or boiled food products that are fermented, or not fermented, or leavened with a chemical yeast (for example sodium bicarbonate). Among the fermented products, there are products fermented with yeasts, or from mixed fermentation. In the first case we find everyday bread (in turn subdivided between immediate fermentation and deferred fermentation), or pizzas, donuts, sweets, cakes, depending on the recipe formula. In the second case we find sourdough bread, or sweet products, such as panettone, pandoro, or others. Among the unfermented products there is unleavened bread, piadinas, tortillas or suchlike. Among the products leavened with chemical yeast there are cakes, sweet pies or savory pies. According to one embodiment, the baking can take place according to conventional recipe, such as at a temperature between 100° C. and 300° C., in particular between 150° C. and 280° C., for a time comprised between 5 and 70 minutes, in particular between 10 and 60 minutes, possibly also based on the size of the dough or each portion of dough. The invention does not rest in amending the recipe, but in replacing traditional wheat flour with the ketogenic flour blend, to ultimately obtain a baked or boiled food product compliant with the subject's ketogenic diet.

The blend is preferably packaged in the form of sachets or pouches. Given the high amount of oil, it is preferred that the blend is provided in a pouch, preferably a stand-up pouch or ‘doypack’, preferably designed with laminated aluminum, and wherein the bottom part of the stand-up pouch is gusseted to provide support for display and use. The blend is preferably provided in a shelf-stable form.

In accordance with the above purposes, the invention also relates to a method to produce a baked or boiled food product, which baked or boiled food product has a ketogenic ratio compliant to the subject's ketogenic regimen. The terms ‘ketogenic regimen’ and ‘ketogenic diet’ are used interchangeably, and typically refer to a category of diets intended to promote the formation of ketone bodies or ketosis in the human subject. In accordance with one embodiment, the method provides to make available a dough comprising at least flour and a liquid substance, comprising water, and to obtain from the dough a semi-worked product able to be subjected to baking so as to obtain the baked or boiled food product. In some embodiments, the dough can comprise other substances or ingredients, useful for the formulation to obtain the baked or boiled food product, and such other substances or ingredients can be enzymes to promote leavening, for example amylases, and/or stabilizing substances, and/or emulsifying substances and/or yeasts and/or salt, and/or combinations thereof. Yeasts have their traditional function of promoting the leavening of the food formulation before cooking in the oven. The yeasts may comprise brewer's yeast and/or chemical yeast, or combinations thereof.

The method for producing a baked or boiled food product according to the invention comprises providing a dough by mixing the flour blend according to the invention with optional additional ingredients such as egg, butter and baking powder according to the desired or traditional baked or boiled food product recipe, and subjecting the dough to cooking in the oven. In other words, the invention relates to a method for producing a baked or boiled food product following traditional recipe, provided that the ketogenic flour blend is the sole flour component in the recipe. The optional additional ingredients according to the recipe may provide additional fat, protein, and digestible carbohydrates, provided that the dough and the baked or boiled food product retain a ketogenic weight ratio between 2.3:1 and 3.0:1, more preferably between 2.6:1 and 3.0:1. The baked or boiled food product can be any of the list of baked or boiled food products provided here above.

In one embodiment, the semi-worked product obtained from the dough, or the dough itself, can be subjected directly to baking to obtain the desired baked or boiled food product. In another embodiment, the dough and/or the semi-worked product can be subjected to a preservation treatment before being subjected to baking. In this case, the semi-worked product thus preserved can be subjected to baking later. For example, by the term preservation treatment we mean, without this being a limitation to the field of protection, freezing, deep-freezing or sudden lowering of the temperature.

The invention also pertains to a method for providing nutrition to a human subject adhering to a ketogenic diet regimen, comprising administering to the subject a baked or boiled food product as defined herein. Worded differently, the invention relates to the use of the ketogenic blend in the manufacture of a baked or boiled food product for a human subject adhering to a ketogenic diet, wherein the baked or boiled food product has a ketogenic weight ratio between 2.3:1 and 3.0:1, preferably between 2.6:1 and 3.0:1. The baked or boiled food product can be any of the list of baked or boiled food products provided here above. The baked or boiled food product is preferably a home-made baked or boiled food product, which typically is associated with shorter cooking times and reduced cooking temperatures. Also, the invention relates to a baked or boiled food product as defined here above, for use in treatment of a human subject adhering to a ketogenic diet. A human subject adhering to a ketogenic diet or regimen preferably consumes ketogenic compositions exclusively.

In one embodiment, the human subject is adhering to a ketogenic diet for weight management, for example a healthy person using ketogenic diet for losing weight purposes, for example an adult person having a body mass index (BMI) between 19 and 25, or an overweight person having a BMI between 25 and 30, or an obese person having a BMI above 30.

However, the ketogenic blend of the invention is particularly useful for the treatment of patients at increased risk of or suffering from epilepsy, associated with the improved patient's compliance to the ketogenic diet. Hence, the ketogenic composition according to the invention is beneficial for therapeutic use in the treatment of epilepsy and/or prevention of progression of epilepsy in a human subject adhering to a ketogenic diet regimen, and beneficial for therapeutic use in the treatment and/or prevention and/or reduction of the severity of epilepsy and in prevention and/or delay of the formation of epileptiform circuits in the brain, including reduction of the occurrence of epilepsy-associated seizures and reducing the number of epilepsy-associated seizures in a human subject adhering to a ketogenic diet regimen. In addition to the beneficial therapeutic use in epilepsy, the ketogenic composition is beneficial for use in preventing abnormal elevations of blood triglyceride levels, fatty liver development and/or metabolic syndrome development in a human subject adhering to the ketogenic diet. The therapeutic benefits comprise preventing and/or treating behavioural consequences of epilepsy. These subjects benefit from adhering to a ketogenic diet or regimen, and the compositions provided by the inventors improve adherence to such regimen.

In one embodiment, the composition is for use in enhancement of mitochondrial function and reduced use of glucose as a metabolic substrate, and may convey neuroprotective benefits and neuroprotective benefits and provide efficient energy sources in neurodegenerative disorders such as Alzheimer's disease, Parkinson's disease and Amyotrophic lateral sclerosis which are associated with mitochondrial dysfunction and metabolic abnormalities, in a human subject adhering to a ketogenic diet regimen. Reference is made to Elizabeth Neal “Ketogenic dietary therapy for other conditions” (source: https://www.matthewsfriends.org/keto-therapies/keto-therapies-for/other-conditions/). This is part of the skilled person's common general knowledge as evidenced by reviews like Tao, Y., Leng, S. X. and Zhang, H. (2022) “Ketogenic diet: an effective treatment approach for neurodegenerative diseases.” Current neuropharmacology, 20(12), p. 2303, and Jiang, Z., Yin, X., Wang, M., Chen, T., Wang, Y., Gao, Z. and Wang, Z. (2022) “Effects of ketogenic diet on neuroinflammation in neurodegenerative diseases” Aging and disease, 13(4), p. 1146, their contents herewith incorporated by reference Other references (1)-(7) in support to these health effects are listed at the end of this section, their contents herewith incorporated by reference. In a preferred aspect the ketogenic blend is useful for subjects adhering to a ketogenic diet suffering from conditions that benefit from a ketogenic diet including subjects suffering from or at risk of seizures, absences, refractory epilepsy, migraine. The ketogenic blend is also useful for subjects that benefit from a ketogenic diet having attention deficit hyperactivity disorder (ADHD), autism spectrum disorders, oncological disorders such as neuro- and glioblastoma, Alzheimer's disease or age-related mild-cognitive impairment.

The ketogenic blend is also useful for subjects adhering to a ketogenic diet suffering from conditions that benefit from a ketogenic diet including subjects suffering from psychiatric disorders such as autism, depression, anxiety, and schizophrenia; reference is made to Tillery et al. “The use of the ketogenic diet in the treatment of psychiatric disorders” Met. Health Clin. 2021 May; 11(3): 211-219. This review reports that there is a need for more data to clearly define the specific benefits the ketogenic diet may provide, but a positive response in individuals who are able to remain on the diet was demonstrated. The present ketogenic blend is particularly strong in improving adherence to the diet.

Worded differently, the invention pertains to the use of the ketogenic composition according to the invention for the treatment of epilepsy and/or prevention of progression of epilepsy in a subject suffering from epilepsy or at risk of seizures. The invention additionally pertains to the use of the ketogenic composition for preventing abnormal elevations of blood triglyceride levels, fatty liver and/or metabolic syndrome development in a subject suffering from epilepsy or at risk of seizures. The invention further pertains to the use of the ketogenic composition in a subject suffering from epilepsy or at risk of seizures. Worded differently, the invention also relates to a (non-therapeutic) method for treating a human being for any of the above therapeutic indications, and to the use of the ketogenic composition of the invention as described here above, in the manufacture of products for the treatment of any of the above therapeutic indications. The treatment is particularly directed to drug-resistance epilepsy.

### LIST OF REFERENCES

1. Fortier M, Castellano C A, St-Pierre V, Myette-Côté E, Langlois F, Roy M, Morin M C, Bocti C, Fulop T, Godin J P, Delannoy C, Cuenoud B, Cunnane S C. A ketogenic drink improves cognition in mild cognitive impairment: Results of a 6-month RCT. Alzheimers Dement. 2021 March; 17(3):543-552;
2. McDonald T J W, Cervenka M C. The Expanding Role of Ketogenic Diets in Adult Neurological Disorders. Brain Sci. 2018 Aug. 8; 8(8):148;
3. Brockhoff J D, Bereswill S, Heimesaat M M. The impact of ketogenic diet on the onset and progression of multiple sclerosis. Eur J Microbiol Immunol (Bp). 2023 Sep. 4; 13(2):29-36;
4. Dal Bello, S.; Valdemarin, F.; Martinuzzi, D.; Filippi, F.; Gigli, G. L.; Valente, M. Ketogenic Diet in the Treatment of Gliomas and Glioblastomas. Nutrients 2022, 14,3851;
5. LiQ, Liang J, Fu N, Han Y and Qin J (2021) A Ketogenic Diet and the Treatment of Autism Spectrum Disorder. Front. Pediatr. 2021 May 11:9:650624;
6. Pietrzak, D.; Kasperek, K.; Re ̧kawek, P.; Piatkowska-Chmiel, I. The Therapeutic Role of Ketogenic Diet in Neurological Disorders. Nutrients 2022, 14, 1952;
7. Phillips, M. C., Johnston, S. E., Simpson, P., Chang, D. K., Mather, D. and Dick, R. J. (2024) “Time-restricted ketogenic diet in amyotrophic lateral sclerosis: a case study. Frontiers in Neurology, 14, p. 1329541.

The invention is illustrated by the below examples, without being limited thereby.

### EXAMPLES

In the examples, calories are calculated based on Atwater 9-4-4 principles for lipid, carbohydrates and protein, respectively.

### Example 1—Gluten-Free Ketogenic Flour Blend with KD Ratio of 2.6:1

| | 100 gr | Unit |
|---|---|---|
| | 667/2604 | Kcal/kJ |
| Carbohydrates | 11 | G |
| Total sugar | 2.3 | G |
| Added sugar | 2.1 | G |
| Protein | 12 | G |
| Total fat: | 60 | G |
| Polyunsaturated | 10 | G |
| linoleic acid (n6) | 8.5 | |
| α-linolenic acid (n3) | 0.9 | |
| Saturated | 35 | G |
| MCT | 17 | |
| caprylic acid (C8) | 9.8 | |
| capric acid (C10) | 6.2 | |
| Monounsaturated | 15 | G |
| Trans | 0 | G |
| Cholesterol | 0 | G |
| Fibre: | 11 | G |
| Soluble | 4 | G |
| Of which FOS | 3.4 | |
| Insoluble | 7 | |
| Sodium | 452 | Mg |

The blend comprised 4.1 wt % almond flour, 3.5 wt % coconut flour and 1.8 wt % buckwheat flour.

### Comparative Example 2—Stability and Sensory Analysis

A premix flour formulation with a lower ketogenic ratio and comprising MCT was prepared, details provided in the table below; the ingredients used for the formulation were based on oil powder 80%, MCT 70%, almond flour, coconut flour, buckwheat flour, coconut milk powder, resistant starch RS4, albumin powder, psyllium, xanthan gum, citric acid. The composition contained about 0.8 wt % ground flaxseed. The formulation was assessed in terms of sensory attributes for a muffin, where the experience of use is simple, in accordance with food preparation customs.

| | Premix |
|---|---|
| Ingredients\hat{ }\hat{ } | Percentage (%) |
| OIL POWDER 80% * | 46 |
| MCT 70% * | 24.8 |
| ALMOND FLOUR ** | 3.5 |
| COCONUT FLOUR ** | 3.5 |
| COCONUT MILK POWDER ** | 5.5 |
| GROUND FLAXSEED | 0.8 |
| FOS | 3.5 |
| BUCKWHEAT FLOUR \hat{ } | 1.8 |
| RESISTANT STARCH VERSAFIBE | 7 |
| 1490 INGREDION | |

* Available with FrieslandCampina, the Netherlands.
** Available with Vialares SAC, New Garden.
\hat{ } Available with Celidarina, New Garden.
\hat{ }\hat{ }It further comprised about 1-2 wt % of albumin powder, and 2 wt % of xanthan gum and psyllium. Salt, citric acid and sweetener are present in small amounts.

In terms of the theoretical nutritional values, the premix provided the following, per 100 g:

| | Premix | |
|---|---|---|
| Energy value | 640 | kcal |
| Carbohydrates | 9.2 | g |
| Proteins | 12 | g |
| Total fat | 61 | g |
| Dietary fibre | 13 | g |
| Ketogenic ratio | 2.9 | |
| MCT | 17.4 | g |
| FOS | 3.5 | g |

A muffin was made from the premix using the following recipe:

| | Muffin formula | | |
|---|---|---|---|
| Ingredients | g | % | Method of production |
| Premix | 100 | 48.06 | Weighing ingredients |
| Fresh egg | 50 | 24.03 | Separate the yolk from the white. |
| Butter | 28 | 13.46 | On the one hand, beat the premix, |
| Water | 25 | 12.01 | egg yolk, baking powder, butter |
| Baking powder | 5 | 2.40 | and water by hand or with a mixer |
| Stevia | 0.075 | 0.04 | for 2 to 3 minutes until |
| | | | homogenised. |
| | | | On the other hand, beat the egg |
| | | | white until stiff. |
| | | | Add the beaten egg whites to the |
| | | | other mixture with a gentle |
| | | | folding motion. |
| | | | Place in mould |
| | | | Bake for 15 minutes at 170 °C |
| | | | (for small pieces). |

The experiment was repeated with bread and biscuits, and similar results were reported (not shown).

Similar benefits were also found with pasta making use of the ketogenic blend in accordance with the recipe of example 2.

The nutrient content of the muffin was calculated theoretically and presented in the table below:

| MUFFIN | | |
|---|---|---|
| Energy value | 442 kcal = 1851 kJ | |
| Carbohydrates | 5.9 | g |
| Proteins | 8.6 | g |
| Total fat | 43 | g |
| Dietary fibre | 6.0 | g |

| | |
|---|---|
| Ketogenic ratio | 2.97 |

The formulation was compared to the commercial flour product Cetomix ketoflour (B-life), and to a combination of a ‘ketogenic 4:1 formula’ with fat blend of DHA, ARA, LCP (long-chain polyunsaturated fatty acids other than DHA, ARA), whey protein to which wheat flour was added; it did not contain MCT. The Cetomix ketoflour comprises, per 100 g, 516 kcal, 5 g digestible carbohydrates and 15 g fibers, 8 g protein and 52 g lipid. It did not contain MCT and FOS.

Thereto a muffin was prepared according to the method and recipe described above and compared to a muffin prepared using the above ketogenic 4:1 formula with wheat flour, and Cetomix, using the same amounts of ingredient (mix, fresh egg, butter, water, baking powder, Stevia) as in the table here above.

In terms of the theoretical nutritional values, the premix of the invention, Cetomix and the 4:1 ketogenic formula provided the following, per 100 g:

| | | | | | 4:1 ketogenic | |
|---|---|---|---|---|---|---|
| | Premix of the | | | | composition + | |
| | invention | | Cetomix * | | wheat flour | |
| Energy value | 640 | kcal | 516 | kcal | 704 | kcal |
| Dig. carbohydrates | 9.2 | g | 5 | g | 2.8 | g |
| Proteins | 12 | g | 8 | g | 14.3 | g |
| Total fat | 61 | g | 52 | g | 69.2 | g |
| Dietary fibre | 13 | g | 15 | g | 5.4 | g |

| Ketogenic ratio | 2.9 | | 4.0 | 4.0 | |
|---|---|---|---|---|---|
| MCT | 17.4 | g | 0 | 0 | |
| FOS | 3.5 | g | 0 | 1.5 | g |

* based on information that is commercially available.

Based on a sensory analysis with multiple testers, the three products were categorized as 1-2-3, with a 1 score being the best:

### Sensory Quality Rating Scale

| | ASSIGNED |
|---|---|
| CATEGORY | SCORE |
| Consistency with the pre-established | 5 |
| sensory requirement | |
| Minimal deviation from the pre-set | 4 |
| sensory requirement | |
| Perceptible deviation from the preset | 3 |
| sensory requirement | |
| Significant deviation from the pre-established | 2 |
| sensory requirement | |
| Very considerable deviation from the | 1 |
| pre-established sensory | |

| | Premix | Cetomix | ketogenic 4:1 formula + wheat flour |
|---|---|---|---|
| Tester 1 | 1 | 2 | 3 |
| Tester 2 | 1 | 2 | 3 |
| Tester 3 | 1 | 3 | 2 |
| Tester 4 | 1 | 3 | 2 |

The premix recipe stood out advantageously—in terms of texture, flavor and handling—from commercial products. Small black spots were observed in the premix blend. This was attributed to the use of 0.8 wt % flaxseed oil in the recipe. The inclusion of flaxseed oil was not found to affect the sensory results.

### Example 3: Product According to the Invention; Sensory Testing

Based on example 2, a new formulation was prepared, packed in tri-laminated pouch and stored at room temperature. Flaxseed oil was not used, instead the amount of almond flour was slightly increased to about 4.2-4.3 wt %.

The theoretical nutritional value was calculated as follows:

| | Premix - 100 g | |
|---|---|---|
| Energy value | 640 | kcal |
| Carbohydrates | 9.2 | g |
| Proteins | 12 | g |
| Total fat | 61 | g |
| Dietary fibre | 13 | g |

| | |
|---|---|
| Ketogenic ratio | 2.9 |
| % MCT | 17.4 |
| % FOS | 3.5 |

Biscuits were prepared using the premix:

| | Formula | | |
|---|---|---|---|
| Ingredients | G | % | Recipe |
| Premix | 100 | 69.44 | Weighing ingredients |
| Fresh egg | 30 | 20.83 | Mix the two dry ingredients plus the |
| Butter | 10 | 6.94 | egg and the butter until a |
| Baking powder | 4 | 2.78 | homogeneous dough is formed. |
| Stevia | — | — | Spread a little premix on a counter, |
| | | | place the dough on it and spread a |
| | | | little premix on the dough again to |
| | | | dry the surface of the dough. |
| | | | Roll out the dough until it is about |
| | | | 1 mm thick and cut into the shape |
| | | | of your choice. |
| | | | Cook for 6 minutes at 185 °C. |

With regard to sensory testing, the following scales were used:

The samples were presented in monadic form, coded with a 3-digit random number. The evaluation was carried out on an individual basis.

### Sensory Testing Premix and Biscuit at Different Time Points Up to 8 Months

| | Initial T = 0 | T = 1 | T = 2 | T = 3 | T = 4 | T = 6 | T = 8 |
|---|---|---|---|---|---|---|---|
| Premix | | | | | | | |
| Colour | 5.0 | 5.0 | 4.8 | 4.5 | 4.9 | 4.8 | 4.9 |
| General | 5.0 | 4.8 | 4.8 | 4.6 | 4.8 | 4.4 | 4.7 |
| appearance | | | | | | | |
| odour | 5.0 | 4.7 | 4.6 | 4.3 | 4.5 | 4.3 | 4.1 |
| Biscuit | | | | | | | |
| General | 5.0 | — | 4.9 | — | 5.0 | 4.6 | 5.0 |
| appearance | | | | | | | |
| Odour | 5.0 | — | 5.0 | — | 4.7 | 4.4 | 4.9 |
| Fracturability | 5.0 | — | 4.9 | — | 4.6 | 4.2 | 4.3 |
| Flavour | 5.0 | — | 4.9 | — | 4.6 | 4.2 | 4.6 |
| characteristics | | | | | | | |

### Comparative Example 4—Effect of MCTs

In order to assess the effect of MCTs in terms of sensory and nutritional impact, a recipe with, per 100 g dry weight, 632 kcal, 11 g digestible carbohydrates, 12 g proteins, 60 g total lipids, 11 g dietary fibre, including 2.4 g FOS, was prepared. The ketogenic ratio was 2.6, and the composition contained 17.4 g MCT per 100 g dry weight. This was called ‘recipe 2’. Two recipe variations were prepared; the amount of MCTs was adjusted to 3.2 g/100 g dry weight (‘recipe 1’, with low MCTs) and 21 g/100 g dry weight (‘recipe 3’ with high MCTs), where the ketogenic ratio was maintained at about 2.6. In order to maintain the ketogenic ratio with this variable amount of MCTs minimal adjustment of the amounts of other lipids, digestible carbohydrates and protein content had to be made. The amounts of calories, lipid, protein and fibre (including FOS) were all within acceptable range.

For the tasting, two culinary preparations were prepared, biscuits and muffins.

### (i) Biscuit Preparation Using the Premix According to Recipes 1, 2 and 3:

| | Formula | | |
|---|---|---|---|
| Ingredients | G | % | Recipe |
| Premix | 100 | 69.44 | Weighing ingredients |
| Fresh egg | 30 | 20.83 | Mix the two dry ingredients plus the |
| Butter | 10 | 6.94 | egg and the butter until a homogeneous |
| Baking powder | 4 | 2.78 | dough is formed. |
| Stevia | — | — | Spread a little premix on a counter, |
| | | | place the dough on it and spread a |
| | | | little premix on the dough again to |
| | | | dry the surface of the dough. |
| | | | Roll out the dough until it is about |
| | | | 1 mm thick and cut into the shape |
| | | | of your choice. |
| | | | Cook for 6 minutes at 185 °C. |

Based on a sensory analysis with multiple testers, the three products (biscuits) were categorized as 1-2-3, with a 1 score being the best:

| | Recipe 1 | Recipe 2 | Recipe 3 |
|---|---|---|---|
| Tester 1 | 2 | 1 | 3 |
| Tester 2 | 1 | 3 | 2 |
| Tester 3 | 2 | 1 | 3 |
| Tester 4 | 1 | 2 | 3 |

### (ii) Muffin Preparation Using the Premix According to Recipes 1, 2 and 3:

| | Muffin formula | | |
|---|---|---|---|
| Ingredients | g | % | Method of production |
| Premix | 100 | 48.06 | Weighing ingredients |
| Fresh egg | 50 | 24.03 | Separate the yolk from the white. |
| Butter | 28 | 13.46 | On the one hand, beat the premix, |
| Water | 25 | 12.01 | egg yolk, baking powder, butter |
| Baking powder | 5 | 2.40 | and water by hand or with a mixer |
| Stevia | 0.075 | 0.04 | for 2 to 3 minutes until |
| | | | homogenised. |
| | | | On the other hand, beat the egg |
| | | | white until stiff. |
| | | | Add the beaten egg whites to the |
| | | | other mixture with a gentle |
| | | | folding motion. |
| | | | Place in mould |
| | | | Bake for 15 minutes at 170 °C |
| | | | (for small pieces). |

Based on a sensory analysis with multiple testers, the three products (muffins) were categorized as 1-2-3, with a 1 score being the best:

| | Recipe 1 | Recipe 2 | Recipe 3 |
|---|---|---|---|
| Tester 1 | 3 | 1 | 2 |
| Tester 2 | 2 | 1 | 3 |
| Tester 3 | 1 | 2 | 3 |
| Tester 4 | 3 | 1 | 2 |

During the preparation of biscuits and muffins, recipe 3, with higher MCT, had a lack of consistency, elasticity, crunchiness and increased oiliness in mouths and hands. Recipe 3 received a lower panel score in both muffin and biscuit preparations.

As a result of tasting, the conclusion was that for biscuits, recipes 1 and 2 yielded similar results. However, for the muffins, recipe 2 was clearly the favorite one. Hence, due to a better overall score, compared to recipe 1, recipe 2 could be adapted to different preparations which improves adherence to a ketogenic diet, and it also had a higher amount of MCTs which is deemed favorable for the patient's pathology. Hence, recipe 2 with 17.4% MCT was preferred based on performance during preparations, sensory profile and nutritional profile.

### Example 5

The premix of example 1 was given to a number of subjects to prepare cheese breadsticks using the following recipe, and compare it to cheese breadsticks prepared by Cetomix which they used previously:
premix* 50 gr
Sardo cheese 30 gr
Ciboulette 5 gr
olive oil 15 cc

### Method of Production:

### Weighing Ingredients

In a bowl, combine premix, sardo cheese, chopped chives, and oil. Add cold water (as needed) and mix until a homogeneous dough is obtained.
Place a silicone sheet on a baking tray.
Pour the mixture onto the tray with a spoon and roll it out with a rolling pin until you achieve a flat surface, giving it the desired thickness.
Sprinkle grated cheese over the dough (optional).
Bake in a preheated oven for 10 minutes or until golden brown.
Remove and, while still hot, cut into sticks. Then, let them cool to become crispy.

They were asked to report back on the breadsticks prepared by the new and old recipes, and in each and every case (n=19) the breadsticks prepared with recipe 1 were favored (100% superiority). Details of the test results for the premix are provided below:

| | |
|---|---|
| Flavor likability | 100% (19) |
| Tolerance | 95% (18) |
| Superiority vs other pre-mix formulas available | 100% (19) |
| Attributes (score 1 to 5) 5 excellent | |
| Crunchiness | 4.84 |
| Versatility | 4.63 |
| Taste | 4.89 |
| Texture | 4.78 |
| Without oily aftertaste | 4.84 |

1. A substantially gluten-free ketogenic flour blend having a ketogenic weight ratio between 2.3:1 and 2.9:1, preferably between 2.4:1 and 2.9:1, the blend comprising between 50 and 70 gram lipid per 100 g dry weight, comprising at least one, more preferably at least two, most preferably all of: (a) between 15 and 20 g MCT/100 g dry weight; (b) MCT providing 20-30% of the total energy content of the blend; (c) between 20 and 35 wt % MCT based on total weight of the lipid, and further comprising at least 2.5 g/100 g dry weight of fructo-oligosaccharides (FOS) and/or galacto-oligosaccharides (GOS), preferably FOS.

2. The flour blend according to claim 1, comprising a mixture of almond flour and/or coconut flour, and buckwheat flour, preferably comprising less than 0.5 wt % flaxseed flour, based on the total dry weight of the blend.

3. The flour blend according to any one of the preceding claims, comprising protein and digestible carbohydrates in a weight ratio between 1.2:1 and 1:1.2.

4. The flour blend according to any one of the preceding claims, comprising between 55 and 65 wt % lipid, based on total dry weight.

5. The flour blend according to any one of the preceding claims, comprising 20-35 wt %, more preferably 20-30 wt %, even more preferably 25-30 wt % MCT, based on total weight of the lipid fraction.

6. The flour blend according to claim 5, wherein the MCT provide 20-30 en % of the total caloric content of the blend.

7. The flour blend according to any one of the preceding claims, comprising 3-5 g/100 g dry weight of GOS and/or FOS, preferably FOS.

8. The flour blend according to any one of the preceding claims, providing 6.0-7.0 kcal/g dry weight of the blend.

9. The flour blend according to any one of the preceding claims, comprises 7-15 g dietary fibers, preferably 8-14 g, more preferably 10-12 g fibers per 100 g of the ketogenic blend.

10. The flour blend according to any one of the preceding claims, comprising 10-20 g of protein per 100 g, preferably 10-15 g of protein per 100 g of the blend; and/or wherein the protein provides 5-10 en % of the total energy content of the ketogenic blend; and/or comprising 10-20 g of digestible carbohydrates per 100 g, preferably 10-15 g of digestible carbohydrates per 100 g of the blend; and/or wherein the digestible carbohydrates provide 5-10 en % of the total energy content of the ketogenic blend.

11. The flour blend according to any one of the preceding claims, comprising casein and whey protein, almond flour, coconut flour, buckwheat flour, coconut milk, fructo-oligosaccharides, Psyllium, xanthan gum, resistant starch and albumin, preferably providing, per 100 g,

| | 667 kcal/2604 kJ | |
|---|---|---|
| digestible carbohydrates | 11 | g |
| total sugar | 2.3 | g |
| added sugar | 2.1 | g |
| Protein | 12 | g |
| total fat: | 60 | g |
| polyunsaturated | 10 | g |
| linoleic acid (n6) | 8.5 | |
| α-linolenic acid (n3) | 0.9 | |
| Saturated | 35 | g |
| MCT, of which: | 17 | |
| caprylic acid (C8) | 9.8 | |
| capric acid (C10) | 6.2 | |
| Monounsaturated | 15 | g |
| Fibre: | 11 | g |
| soluble, | 4 | g, |
| of which FOS | 3.4 | |
| Insoluble | 7 | |

allowing for a 10% variation in the above numbers, provided that the sum of carbohydrates, protein, fat and fibre amounts to 100%, and the blend has a ketogenic weight ratio between 2.4:1 and 2.9:1, and wherein the blend comprises a mixture of almond flour, coconut flour, and buckwheat flour, the latter preferably in an amount of less than 50% of all flour ingredients comprised in the blend.

12. A method for producing a bakery product, comprising providing a dough by mixing the flour blend according to any one of the preceding claims with egg, butter and baking powder according to the desired bakery product recipe, and subjecting the dough to baking.

13. A bakery product suitable for a ketogenic diet and obtainable by the method according to claim 12, preferably having a ketogenic weight ratio between 2.4:1 and 3.0:1, more preferably between 2.6:1 and 3.0:1.

14. A (non-therapeutic) method for providing nutrition to a human subject adhering to a ketogenic diet regimen, comprising administering to the patient a bakery product according to claim 13.

15. A substantially gluten-free ketogenic flour blend according to any one of claims 1-11, for use in the treatment of epilepsy and/or prevention of progression of epilepsy in a human subject adhering to a ketogenic diet regimen, for use in the treatment and/or prevention and/or reduction of the severity of epilepsy and in prevention and/or delay of the formation of epileptiform circuits in the brain in a human subject adhering to a ketogenic diet regimen, and/or for use in preventing abnormal elevations of blood triglyceride levels, fatty liver development and/or metabolic syndrome development in a human subject adhering to a ketogenic diet; and/or for use in enhancement of mitochondrial function and reduced use of glucose as a metabolic substrate, particularly Alzheimer's disease, Parkinson's disease and Amyotrophic lateral sclerosis in a human subject adhering to a ketogenic diet regimen; and/or for use in treatment of human subjects suffering from psychiatric disorders such as autism, depression, anxiety, and schizophrenia, wherein the human subject adheres to a ketogenic diet regimen.
